(19) United States
(12) Patent Application Publication
(10) Pub. No.: US 2024/0182841 A1
(43) Pub. Date: Jun. 6, 2024

(54) HIGHLY PRACTICAL CRYOVIAL

(71) Applicant: Meidong Huicheng Life Technology (Kunshan) Co., Ltd., Suzhou, Jiangsu (CN)

(72) Inventors: ZIXIAN LIU, Suzhou, Jiangsu (CN); YIYONG YUAN, Suzhou, Jiangsu (CN); SHU YANG, Suzhou, Jiangsu (CN); GANG LIU, Suzhou, Jiangsu (CN); HUA ZHAO, Suzhou, Jiangsu (CN); AIZE SUN, Suzhou, Jiangsu (CN)

(21) Appl. No.: 17/796,212

(22) PCT Filed: Nov. 9, 2021
(86) PCT No.: PCT/CN2021/129613

§ 371 Date: Jul. 28, 2022

(30) Foreign Application Priority Data
Oct. 1, 2021 (CN) 202111167038.4

(51) Int. Cl. C12M 1/34; C12M 1/00; C12M 1/24; C12M 1/26

(52) U.S. Cl. CPC C12M 41/12; C12M 23/08; C12M 23/38; C12M 33/04

(57) ABSTRACT

The present disclosure provides a highly practical cryovial, which relates to the technical field of the cryovial. The cryovial comprises an inner tube, a transparent outer tube, a limit snap ring, a liquid control mechanism arranged inside the inner tube, a protection mechanism arranged on the transparent outer tube, and a sealed liquid taking mechanism arranged on the inner tube, wherein the liquid control mechanism comprises a push rod and a piston block which is movably connected with the inner tube, first spiral holes of the same size are formed at the corresponding positions of the bottom ends of the inner tube and the transparent outer tube, and the push rod is movably connected with the two first spiral holes respectively.

### TECHNICAL FIELD

The present disclosure relates to the technical field of a cryovial, and in particular to a highly practical cryovial.

### BACKGROUND

A cryovial is also called a strain preservation tube, a magnetic bead preservation tube, or a magnetic bead cryovial. The commonly used strain preservation methods in a microbiology laboratory include a milk method, a glycerin method, a slant method, etc., with different degrees of complexity and different effects. Generally, the cryovial can be roughly divided into an inner tube and a tube cover. The inner tube is generally made of medical-grade low-temperature resistant polypropylene, and the tube cover is generally made of high-density polyethylene and is mainly used in liquid nitrogen at −196° C. The cryovial mainly provides effective and safe guarantee and convenient management for biological sample warehouses, biological product warehouses, and chemicals requiring low-temperature storage.

Generally, the cryovial is used by three steps: Step I, selecting fresh cultures from pure bacterial cultures to prepare bacterial suspensions with a turbidity of about 3-4 Maxwell's specific turbidity for inoculation in the strain preservation tube; Step II, tightening the preservation tube, and turning it back and forth for 4-5 times to emulsify the bacteria without shaking; and Step III, putting the preservation tube in a refrigerator for preservation, with a temperature usually between −20° C. and −70° C.

The existing cryovial has some problems, such as the uncontrollable amount of liquid taking, lack of necessary protection mechanism for a transparent outer tube, and pollution caused by insufficient sealing during liquid taking. Therefore, we put forward a highly practical cryovial.

### SUMMARY

The present disclosure mainly aims to provide a highly practical cryovial, which can effectively solve the problems in the background art, such as the uncontrollable amount of liquid taking, lack of necessary protection mechanism for a transparent outer tube, and pollution caused by insufficient sealing during liquid taking.

In order to solve the above technical problems, the present disclosure is realized by the following technical solution: a highly practical cryovial, comprising an inner tube, a transparent outer tube, a limit snap ring, a liquid control mechanism arranged inside the inner tube, a protection mechanism arranged on the transparent outer tube, and a sealed liquid taking mechanism arranged on the inner tube, wherein the inner tube and the transparent outer tube are of a cylindrical structure and internally hollow; the liquid control mechanism comprises a push rod and a piston block, wherein the piston block is movably connected with the inner tube, first spiral holes of the same size are formed at the corresponding positions of the bottom ends of the inner tube and the transparent outer tube, and the push rod is movably connected with the two first spiral holes respectively; the protection mechanism comprises an anti-slip pad and a clamping pad, wherein one end of the anti-slip pad is movably connected with an outer side wall of the transparent outer tube and the other end thereof is movably connected with one side of the clamping pad close to the inner tube, a first mounting hole is formed at a central position inside the limit snap ring, and the outer side wall of the inner tube is connected with the first mounting hole; and the sealed liquid taking mechanism comprises a sealing cover, wherein the sealing cover is of a cylindrical structure, internally hollow, and movably connected with the inner tube, and a lower part of the sealing cover is movably connected with an upper part of the limit snap ring.

Preferably, the inner tube further comprises an inner sealing sleeve and a clamping ring, wherein the inner sealing sleeve is arranged above the inside of the inner tube and located at a central position, a second spiral hole is formed at a central position of the inner sealing sleeve, the push rod is movably connected with the second spiral hole, a second mounting hole is formed at a central position of the clamping ring, an outer side wall of the inner tube is connected with the second mounting hole, and the clamping ring is located on one side away from the inner sealing sleeve. The inner sealing sleeve could ensure that the inner tube is in a sealed state.

Preferably, the transparent outer tube further comprises a buffer spacer, an outer sealing sleeve, and a scale line, wherein the buffer spacer is arranged between the inner tube and the transparent outer tube, the outer sealing sleeve is arranged below the outside of the transparent outer tube and located at a central position, a third spiral hole is formed at a central position of the outer sealing sleeve, the push rod is movably connected with the third spiral hole, and the scale line is formed on an outer side wall of the transparent outer tube. The buffer spacer could ensure that the inner tube and the transparent outer tube do not wear each other.

Preferably, the liquid control mechanism further comprises a rotating frame, the push rod is arranged below the piston block and located at a central position, and the other end of the push rod is connected to an upper part of the rotating frame. The piston block could move up and down inside the inner tube by rotating the rotating frame.

Preferably, the protection mechanism further comprises first anti-slip strips, wherein the nineteen first anti-slip strips are all arranged on the side of the clamping pad away from the inner tube and arranged along the clamping pad in an equally divided circumference manner. The first anti-slip strips could increase connection friction between the clamping pad and a placement component to ensure stable placement.

Preferably, the fastening mechanism further comprises second anti-slip strips, a sealing ring, and a liquid taking connector, wherein the twelve second anti-slip strips are all arranged on an outer side wall of the sealing cover and arranged along the sealing cover in an equally divided circumference manner, the sealing ring is of a cylindrical structure and internally hollow, a third mounting hole is formed at a central position of the sealing cover, the outer side wall of the sealing ring is fixedly connected with the third mounting hole, and the other end of the sealing ring is movably connected with the liquid taking connector. The liquid taking connector could facilitate an external liquid taking tube to take and discharge the liquid.

Preferably, the transparent outer tube further comprises a support component which is arranged below the outside of the transparent outer tube and comprises a base, a fixing ring, and support columns, wherein the upper part of the base is connected with a lower part of the outside of the transparent outer tube, the fixing ring is arranged above the base and movably connected with the transparent outer tube, the four support columns are all arranged above the base and arranged along the base in an equally divided circumference manner, and the other ends of the four support columns are all connected with the lower part of the outside of the inner tube. The fixing ring could fix the position of the transparent outer tube.

Preferably, the inner tube further comprises a fastening component which is arranged on the outer side wall of the inner tube and comprises a mounting snap ring and clamping columns, wherein a fourth mounting hole is formed at a central position of the mounting snap ring, the outer side wall of the inner tube is connected with the fourth mounting hole, the four clamping columns are all arranged below the mounting snap ring and arranged in an equally divided circumference manner, and the other ends of the four clamping columns are movably connected with the clamping ring. For the sliding between the clamping columns and the clamping ring, the stable placement between the inner tube and the transparent outer tube is achieved.

The present disclosure has the following beneficial effects:
1. In the present disclosure, the strains are preserved in the inner tube, the content of which is specifically displayed on the scale line. The state of the piston block could be changed by rotating the rotating frame to make it slide up and down inside the inner tube. The change in the position for preserving the strains in the inner tube could facilitate the control of the position of the strains in the inner tube. When taking the strains, the piston block slides in the inner tube to control the taking amount, and the specific taking amount is displayed on the scale line, which could greatly avoid the situation of multiple taking caused by too much or too little taking, thereby enhancing the practicality;
2. In the present disclosure, the transparent outer tube is connected with an external placement plate to place the whole cryovial. As the cryovial itself is fragile, a protective component is added outside the transparent outer tube to enhance the protection. The anti-slip pad could reduce the friction between the transparent outer tube and the clamping pad, and the first anti-slip strips could increase the friction between the clamping pad and the external placement plate, thereby increasing the placement stability and ensuring that the cryovial is in the optimal placement state;
3. In the present disclosure, the movable connection between the sealing cover and the inner tube realizes the opening and sealed storage of the inner tube, and the limit snap ring could prevent the sealing cover from rotating too tightly. For traditional taking, the sealing cover should be opened, and upon improvement, the sealing ring and the liquid taking connector could be used in combination without inserting the liquid taking tube into the inner tube for taking; and the liquid taking tube is movably connected with the liquid taking connector for taking, which is convenient to use, avoids pollution, and greatly enhances the practicability.

Of course, the implementation of any product of the present disclosure does not necessarily achieve all the above advantages at the same time.

### BRIEF DESCRIPTION OF DRAWINGS

A brief introduction is made below to the drawings necessary for the description of the embodiment to illustrate the technical solution in the embodiments of the present disclosure more clearly. Apparently, the figures in the following description are only some embodiments of the present disclosure, and those of ordinary skill in the art can derive other drawings from these drawings without creative work.

FIG. 1 is a schematic diagram for an overall structure of a highly practical cryovial of the present disclosure;

FIG. 2 is a schematic diagram for a front-view structure of a highly practical cryovial of the present disclosure;

FIG. 3 is a schematic diagram for a side-view structure of a highly practical cryovial of the present disclosure;

FIG. 4 is a schematic diagram for a profile structure at A-A in FIG. 2 of the present disclosure;

FIG. 5 is a partially enlarged structural diagram at B in FIG. 4 of the present disclosure.

FIG. 6 is a partially enlarged structural diagram at C in FIG. 4 of the present disclosure.

In the drawings, the list of components represented by each number is as follows:

1. inner tube; 2. transparent outer tube; 3. buffer spacer; 4. base; 5. fixing ring; 6. support column; 7. inner sealing sleeve; 8. outer sealing sleeve; 9. push rod; 10. piston block; 11. rotating frame; 12. anti-slip pad; 13. first antiskid strip; 14. clamping ring; 15. mounting snap ring; 16. clamping column; 17. limit snap ring; 18. sealing cover; 19. second anti-slip strip; 20. sealing ring; 21. liquid taking connector; 22. scale line; 23. clamping pad.

### DETAILED DESCRIPTION

In order to make the technical means, creative features, achievement purpose, and efficacy of the present disclosure easy to understand, the present disclosure will be further explained below in combination with specific embodiments.

It should be noted in the description of the present disclosure that the directions or position relationships such as “upper”, “lower”, “inside”, “outside”, “front end”, “rear end”, “two ends”, “one end”, and “the other end” are based on those shown in the figures, and are used only for facilitating the description of the present disclosure and for simplified description, not for indicating or implying that the target devices or components must have a special direction and be structured and operated at the special direction, therefore they cannot be understood as the restrictions to the present disclosure. Moreover, the words “first” and “second” are used only for description, and cannot be understood as an indication or implication of relative importance.

It should be noted in the description of the present disclosure that unless otherwise specified or restricted, the words “installation”, “provided with”, and “connection” should be understood as a general sense. For example, the “connection” can be fixed connection, removable connection, integrated connection, mechanical connection, electrical connection, direct connection, indirect connection through intermediate media or connection between two components. Persons of ordinary skill in the art can understand the specific meanings of the terms above in the present disclosure as the case may be.

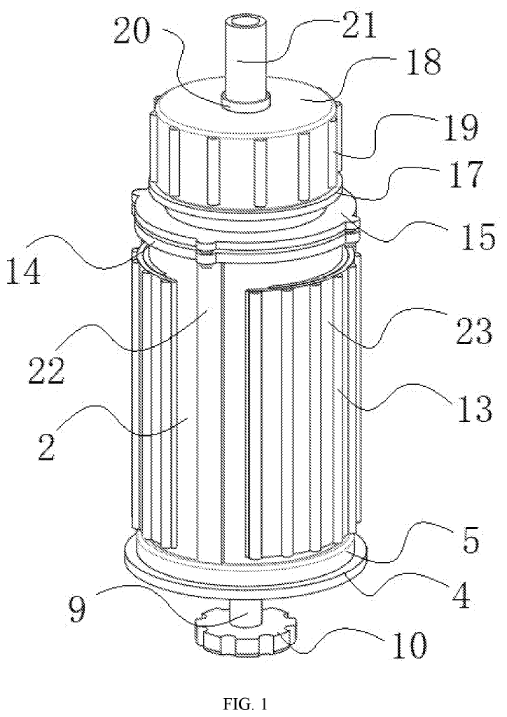

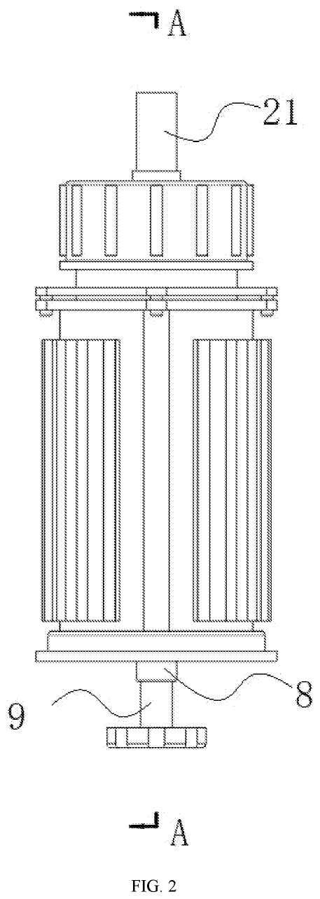

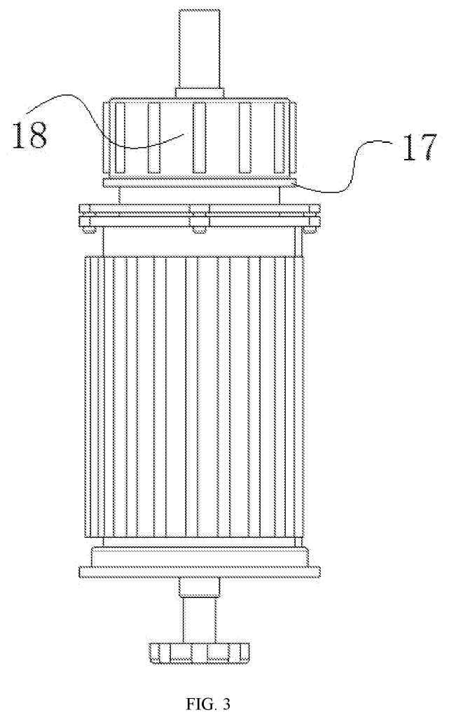

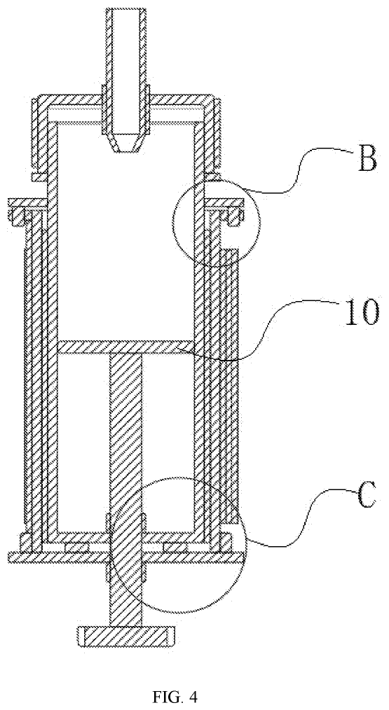

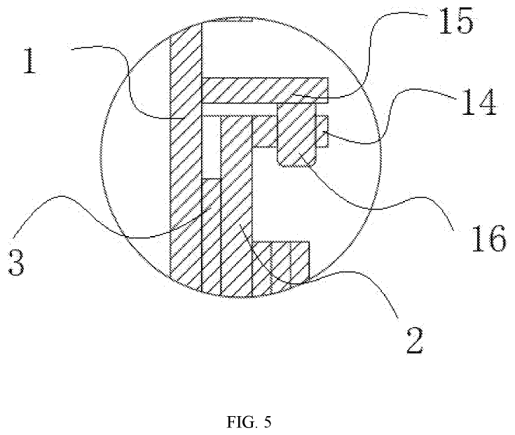

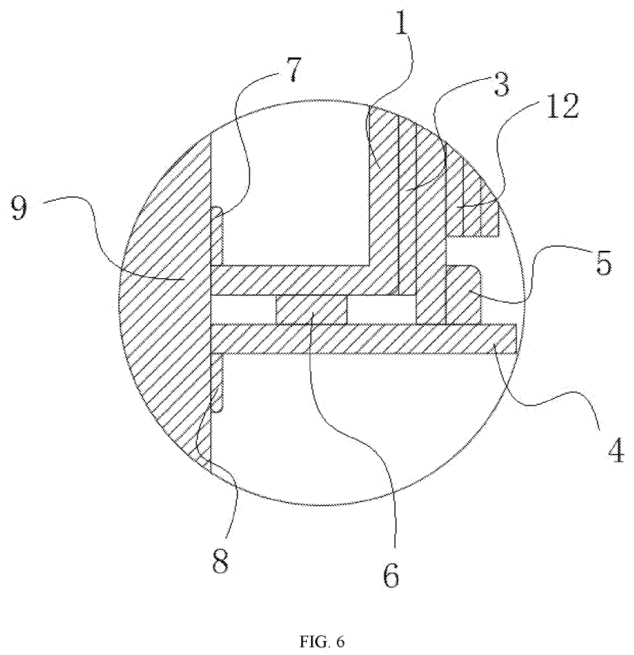

Referring to FIGS. 1-6, the present disclosure is a highly practical cryovial, comprising an inner tube 1, a transparent outer tube 2, a limit snap ring 17, a liquid control mechanism arranged inside the inner tube 1, a protection mechanism arranged on the transparent outer tube 2, and a sealed liquid taking mechanism arranged on the inner tube 1, wherein the inner tube 1 and the transparent outer tube 2 are of a cylindrical structure and internally hollow; the liquid control mechanism comprises a push rod 9 and a piston block 10, wherein the piston block 10 is movably connected with the inner tube 1, first spiral holes of the same size are formed at the corresponding positions of the bottom ends of the inner tube 1 and the transparent outer tube 2, and the push rod 9 is movably connected with the two first spiral holes respectively; the protection mechanism comprises an anti-slip pad 12 and a clamping pad 23, wherein one end of the anti-slip pad 12 is movably connected with an outer side wall of the transparent outer tube 2 and the other end thereof is movably connected with one side of the clamping pad 23 close to the inner tube 1, a first mounting hole is formed at a central position inside the limit snap ring 17, and the outer side wall of the inner tube 1 is connected with the first mounting hole; and the sealed liquid taking mechanism comprises a sealing cover 18, wherein the sealing cover 18 is of a cylindrical structure, internally hollow, and movably connected with the inner tube 1, and a lower part of the sealing cover 18 is movably connected with an upper part of the limit snap ring 17.

Further, the inner tube 1 further comprises an inner sealing sleeve 7 and a clamping ring 14, wherein the inner sealing sleeve 7 is arranged above the inside of the inner tube 1 and located at a central position, a second spiral hole is formed at a central position of the inner sealing sleeve 7, the push rod 9 is movably connected with the second spiral hole, a second mounting hole is formed at a central position of the clamping ring 14, an outer side wall of the inner tube 1 is connected with the second mounting hole, and the clamping ring 14 is located on one side away from the inner sealing sleeve 7. The inner sealing sleeve 7 could ensure that the inner tube 1 is in a sealed state.

Further, the transparent outer tube 2 further comprises a buffer spacer 3, an outer sealing sleeve 8, and a scale line 22, wherein the buffer spacer 3 is arranged between the inner tube 1 and the transparent outer tube 2, the outer sealing sleeve 8 is arranged below the outside of the transparent outer tube 2 and located at a central position, a third spiral hole is formed at a central position of the outer sealing sleeve 8, the push rod 9 is movably connected with the third spiral hole, and the scale line 22 is formed on an outer side wall of the transparent outer tube 2. The buffer spacer 3 could ensure that the inner tube 1 and the transparent outer tube 2 do not wear each other.

Furthermore, the liquid control mechanism further comprises a rotating frame 11, the push rod 9 is arranged below the piston block 10 and located at a central position, and the other end of the push rod 9 is connected to an upper part of the rotating frame 11. The piston block 10 could move up and down inside the inner tube 1 by rotating the rotating frame 11.

Furthermore, the protection mechanism further comprises first anti-slip strips 13, wherein the nineteen first anti-slip strips 13 are all arranged on the side of the clamping pad 23 away from the inner tube 1 and arranged along the clamping pad 23 in an equally divided circumference manner. The first anti-slip strips 13 could increase connection friction between the clamping pad 23 and a placement component to ensure stable placement.

Furthermore, the fastening mechanism further comprises second anti-slip strips 19, a sealing ring 20, and a liquid taking connector 21, wherein the twelve second anti-slip strips 19 are all arranged on an outer side wall of the sealing cover 18 and arranged along the sealing cover 18 in an equally divided circumference manner, the sealing ring 20 is of a cylindrical structure and internally hollow, a third mounting hole is formed at a central position of the sealing cover 18, the outer side wall of the sealing ring 20 is fixedly connected with the third mounting hole, and the other end of the sealing ring 20 is movably connected with the liquid taking connector 21. The liquid taking connector 21 could facilitate an external liquid taking tube to take and discharge the liquid.

Furthermore, the transparent outer tube 2 further comprises a support component which is arranged below the outside of the transparent outer tube 2 and comprises a base 4, a fixing ring 5, and support columns 6, wherein the upper part of the base 4 is connected with a lower part of the outside of the transparent outer tube 2, the fixing ring 5 is arranged above the base 4 and movably connected with the transparent outer tube 2, the four support columns 6 are all arranged above the base 4 and arranged along the base 4 in an equally divided circumference manner, and the other ends of the four support columns 6 are all connected with the lower part of the outside of the inner tube 1. The fixing ring 5 could fix the position of the transparent outer tube 2.

Further, the inner tube 1 further comprises a fastening component which is arranged on the outer side wall of the inner tube 1 and comprises a mounting snap ring 15 and clamping columns 16, wherein a fourth mounting hole is formed at a central position of the mounting snap ring 15, the outer side wall of the inner tube 1 is connected with the fourth mounting hole, the four clamping columns 16 are all arranged below the mounting snap ring 15 and arranged in an equally divided circumference manner, and the other ends of the four clamping columns 16 are movably connected with the clamping ring 14. For the sliding between the clamping columns 16 and the clamping ring 14, the stable placement between the inner tube 1 and the transparent outer tube 2 is achieved.

The present disclosure has the working principle: The strains are preserved in the inner tube 1, the content of which is specifically displayed on the scale line 22. The transparent outer tube 2 is connected with an external placement plate to place the whole cryovial. As the cryovial itself is fragile, a protective component is added outside the transparent outer tube 2 to enhance the protection. The anti-slip pad 12 could reduce the friction between the transparent outer tube 2 and the clamping pad 23, and the first anti-slip strips 13 could increase the friction between the clamping pad 23 and the external placement plate, the support column 6 could ensure that the inner tube 1 is stably placed inside the transparent outer tube 2, and the fixing ring 5 could ensure that the transparent outer tube 2 is stably placed on the base 4, thereby enhancing the placement stability, and ensuring that the cryovial is in the best placement state. The state of the piston block 10 could be changed by rotating the rotating frame 11 to make it slide up and down inside the inner tube 1. The change in the position for preserving the strains in the inner tube 1 could facilitate the control of the position of the strains in the inner tube 1. When taking the strains, the piston block 10 slides in the inner tube 1 to control the taking amount, and the specific taking amount is displayed on the scale line 22. The movable connection between the sealing cover 18 and the inner tube 1 realizes the opening and sealed storage of the inner tube 1, and the limit snap ring 17 could prevent the sealing cover 18 from rotating too tightly. For traditional taking, the sealing cover 18 should be opened, and upon improvement, the sealing ring 20 and the liquid taking connector 21 could be used in combination without inserting the liquid taking tube into the inner tube 1; and the liquid taking tube is movably connected with the liquid taking connector 21 for taking, which is convenient to use, avoids pollution, and greatly enhances the practicability.

The basic principle, main features, and advantages of the present disclosure are shown and described above. Those skilled in the art should know that the present disclosure is not limited by the above embodiments. The above embodiments and description only describe the principles of the present disclosure. Without departing from the spirit and scope of the present disclosure, various changes and improvements will also be made to the present disclosure, and fall within the requested scope of the present disclosure. The scope of protection requested by the present disclosure is defined by the attached claim and its equivalents.

1. A highly practical cryovial, comprising an inner tube (1), a transparent outer tube (2), a limit snap ring (17), a liquid control mechanism arranged inside the inner tube (1), a protection mechanism arranged on the transparent outer tube (2), and a sealed liquid taking mechanism arranged on the inner tube (1), wherein the inner tube (1) and the transparent outer tube (2) are of a cylindrical structure and internally hollow; the liquid control mechanism comprises a push rod (9) and a piston block (10), wherein the piston block (10) is movably connected with the inner tube (1), first spiral holes of the same size are formed at the corresponding positions of the bottom ends of the inner tube (1) and the transparent outer tube (2), and the push rod (9) is movably connected with the two first spiral holes respectively; the protection mechanism comprises an anti-slip pad (12) and a clamping pad (23), wherein one end of the anti-slip pad (12) is movably connected with an outer side wall of the transparent outer tube (2) and the other end thereof is movably connected with one side of the clamping pad (23) close to the inner tube (1), a first mounting hole is formed at a central position inside the limit snap ring (17), and an outer side wall of the inner tube (1) is connected with the first mounting hole; and the sealed liquid taking mechanism comprises a sealing cover (18), wherein the sealing cover (18) is of a cylindrical structure, internally hollow, and movably connected with the inner tube (1), and a lower part of the sealing cover (18) is movably connected with an upper part of the limit snap ring (17).

2. The highly practical cryovial according to claim 1, wherein the inner tube (1) further comprises an inner sealing sleeve (7) and a clamping ring (14), wherein the inner sealing sleeve (7) is arranged above the inside of the inner tube (1) and located at a central position, a second spiral hole is formed at a central position of the inner sealing sleeve (7), the push rod (9) is movably connected with the second spiral hole, a second mounting hole is formed at a central position of the clamping ring (14), the outer side wall of the inner tube (1) is connected with the second mounting hole, and the clamping ring (14) is located on one side away from the inner sealing sleeve (7).

3. The highly practical cryovial according to claim 2, wherein the transparent outer tube (2) further comprises a buffer spacer (3), an outer sealing sleeve (8), and a scale line (22), wherein the buffer spacer (3) is arranged between the inner tube (1) and the transparent outer tube (2), the outer sealing sleeve (8) is arranged below the outside of the transparent outer tube (2) and located at a central position, a third spiral hole is formed at a central position of the outer sealing sleeve (8), the push rod (9) is movably connected with the third spiral hole, and the scale line (22) is formed on an outer side wall of the transparent outer tube (2).

4. The highly practical cryovial according to claim 3, wherein the liquid control mechanism further comprises a rotating frame (11), the push rod (9) is arranged below the piston block (10) and located at a central position, and the other end of the push rod (9) is connected to an upper part of the rotating frame (11).

5. The highly practical cryovial according to claim 4, wherein the protection mechanism further comprises first anti-slip strips (13), wherein the nineteen first anti-slip strips (13) are all arranged on one side of the clamping pad (23) away from the inner tube (1) and arranged along the clamping pad (23) in an equally divided circumference manner.

6. The highly practical cryovial according to claim 5, wherein the fastening mechanism further comprises second anti-slip strips (19), a sealing ring (20), and a liquid taking connector (21), wherein the twelve second anti-slip strips (19) are all arranged on an outer side wall of the sealing cover (18) and arranged along the sealing cover (18) in an equally divided circumference manner, the sealing ring (20) is of a cylindrical structure and internally hollow, a third mounting hole is formed at a central position of the sealing cover (18), the outer side wall of the sealing ring (20) is fixedly connected with the third mounting hole, and the other end of the sealing ring (20) is movably connected with the liquid taking connector (21).

7. The highly practical cryovial according to claim 6, wherein the transparent outer tube (2) further comprises a support component which is arranged below the outside of the transparent outer tube (2) and comprises a base (4), a fixing ring (5), and support columns (6), wherein the upper part of the base (4) is connected with a lower part of the outside of the transparent outer tube (2), the fixing ring (5) is arranged above the base (4) and movably connected with the transparent outer tube (2), the four support columns (6) are all arranged above the base (4) and arranged along the base (4) in an equally divided circumference manner, and the other ends of the four support columns (6) are all connected with the lower part of the outside of the inner tube (1).

8. The highly practical cryovial according to claim 7, wherein the inner tube (1) further comprises a fastening component which is arranged on the outer side wall of the inner tube (1) and comprises a mounting snap ring (15) and clamping columns (16), wherein a fourth mounting hole is formed at a central position of the mounting snap ring (15), the outer side wall of the inner tube (1) is connected with the fourth mounting hole, the four clamping columns (16) are all arranged below the mounting snap ring (15) and arranged in an equally divided circumference manner, and the other ends of the four clamping columns (16) are movably connected with the clamping ring (14).
